(19) United States
(12) Patent Application Publication
(10) Pub. No.: US 2024/0366961 A1
(43) Pub. Date: Nov. 7, 2024

(54) SYSTEMS AND METHODS FOR PHOTOACTIVATING A PHOTOSENSITIZER APPLIED TO AN EYE

(71) Applicant: Avedro, Inc., Waltham, MA (US)

(72) Inventors: Marc D. Friedman, Needham, MA (US); MIkhail Smirnov, North Andover, MA (US); Pavel Kamaev, Lexington, KY (US)

(21) Appl. No.: 18/775,674

(22) Filed: Jul. 17, 2024

Related U.S. Application Data
(63) Continuation of application No. 17/521,276, filed on Nov. 8, 2021, now Pat. No. 12,070,618.
Application No. 17/521,276 is a continuation of application No. 16/384,099, filed on Apr. 15, 2019, now Pat. No. 11,167,149.
Application No. 16/384,099 is a continuation of application No. 15/137,748, filed on Apr. 25, 2016, now Pat. No. 10,258,809.
(60) Provisional application No. 62/152,568, filed on Apr. 24, 2015.
(60) Provisional application No. 62/152,533, filed on Apr. 24, 2015.
(60) Provisional application No. 62/279,951, filed on Jan. 18, 2016.

(51) Int. Cl. A61N 5/06; A61F 9/007; A61F 9/02

(52) U.S. Cl. CPC A61N 5/0624; A61F 9/0079; A61F 9/029; A61N 5/062; A61M 2202/0208; A61M 2205/3368; A61M 2205/36; A61M 2205/52; A61M 2210/0612; A61N 2005/0626; A61N 2005/0648; A61N 2005/0661

(57) ABSTRACT

An antimicrobial treatment system comprises a wearable photoactivation device. The wearable photoactivation device includes a body configured to be positioned on a head of a subject over one or more eyes of the subject. The body includes one or more windows or openings that allow the one or more eyes to see through the body. The body includes one or more photoactivating light sources coupled to the body and configured to direct photoactivating light to the one or more eyes according to illumination parameters. The illumination parameters determine a dose of the photoactivating light that activates, according to photochemical kinetic reactions, a photosensitizer applied to the one or more eyes and generates reactive oxygen species that provide an antimicrobial effect in the one or more eyes, without substantially inducing cross-linking activity that produces biomechanical changes in the one or more eyes.

### CROSS-REFERENCE TO RELATED APPLICATIONS

This application is a continuation of U.S. patent application Ser. No. 17/521,276, filed Nov. 8, 2021, which is a continuation of U.S. patent application Ser. No. 16/384,099, filed Apr. 15, 2019, which is a continuation of U.S. patent application Ser. No. 15/137,748, filed Apr. 25, 2016, which claims the benefit of, and priority to, U.S. Provisional Patent Application No. 62/152,568, filed Apr. 24, 2015, U.S. Provisional Patent Application No. 62/152,533, filed Apr. 24, 2015, and U.S. Provisional Patent Application No. 62/279,951, filed Jan. 18, 2016, the contents of these applications being incorporated entirely herein by reference.

### BACKGROUND OF THE INVENTION

### Field of the Invention

The present disclosure pertains to systems and methods for treating an eye, and more particularly, to systems and methods for activating a photosensitizer applied to an eye during a treatment.

### Description of Related Art

Bacterial keratitis 1s an infection of the cornea caused by bacteria, such as Staphylococcus Aureus and Pseudomonas Aeruginosa. Amoebic keratitis is an infection of the cornea caused by amoeba, such as Acanthamoeba. Fungal keratitis is an infection of the cornea caused by fungi. Such eye infections may cause pain, reduced vision, light sensitivity, and tearing or discharge. If left untreated, these eye infections can cause blindness. Superficial keratitis involves the uppermost layers of the cornea, and after healing, usually leaves no scar on the cornea. On the other hand, deep keratitis affects deeper corneal layers, and after healing, may leave a scar that can affect vision depending on where the scar is located. The treatment of these eye infections may involve the application of an antimicrobial agent to the infected eyes.

### SUMMARY

Some antimicrobial treatments employ photosensitizers to sterilize tissues. In general, when photosensitizers are applied to tissue and exposed to photoactivating illumination, resulting photochemical kinetic reactions can produce antimicrobial agents that place microbes in the tissue under stress and induce an apoptotic or necrotic response in the microbes.

Example antimicrobial treatments may, for instance, employ formulations including various concentrations of riboflavin as a photosensitizer. After a riboflavin formulation is applied to tissue, illumination of the tissue with ultraviolet A (UVA) light in particular results in photochemical kinetic reactions that provide an antimicrobial effect.

According to an example embodiment, an antimicrobial treatment system comprises a wearable photoactivation device. The wearable photoactivation device includes a body that defines a chamber shaped to be positioned over and enclose one or more eyes of a subject. The body includes one or more windows that allow the one or more eyes to see through the body. The wearable photoactivation device includes one or more photoactivating light sources coupled to the body and configured to direct photoactivating light to the one or more eyes according to illumination parameters. The illumination parameters determine a dose of the photoactivating light that activates, according to photochemical kinetic reactions, a photosensitizer applied to the one or more eyes and generates reactive oxygen species that provide an antimicrobial effect in the one or more eyes. The wearable photoactivation device includes an inlet configured to couple the body to an oxygen source. The chamber receives oxygen from the oxygen source via the inlet to modify oxygen conditions in the chamber. The activation of the photosensitizer depends on the oxygen conditions.

The wearable photoactivation device may further include one or more heating elements coupled to the body and configured to generate heat in the chamber according to temperature parameters. The temperature parameters modify, according to photochemical kinetic reactions, the activation of the photosensitizer applied to the one or more eyes and the generation of reactive oxygen species that provide the antimicrobial effect in the one or more eyes.

According to another example embodiment, a wearable antimicrobial treatment device includes a body shaped to be positioned on a head of a subject over one or more eyes. The body includes one or more openings that allow the one or more eyes to see through the body. The wearable antimicrobial treatment device includes one or more photoactivating light sources coupled to the body and configured to direct photoactivating light to the one or more eyes according to illumination parameters. The illumination parameters determine a dose of the photoactivating light that activates, according to photochemical kinetic reactions, a photosensitizer applied to the one or more eyes and generates reactive oxygen species that provide an antimicrobial effect in the one or more eyes. The wearable antimicrobial treatment device includes a plurality of guide light sources coupled at a plurality of positions about the body and configured to direct visible light to the one or more eyes from a plurality of directions. The wearable antimicrobial treatment device includes a controller configured to operate the guide light sources to alternately direct the visible light from each direction according to a sequence wherein the subject is directed to look, with the one or more eyes, in each direction according to the sequence and different respective areas of the one or more eyes are exposed to the photoactivating light from the one or more photoactivating light sources.

According to an additional example embodiment, an antimicrobial treatment system comprises a wearable photoactivation device. The wearable photoactivation device includes a body configured to be positioned on a head of a subject over one or more eyes of the subject. The body includes one or more windows or openings that allow the one or more eyes to see through the body. The body includes one or more photoactivating light sources coupled to the body and configured to direct photoactivating light to the one or more eyes according to illumination parameters. The illumination parameters determine a dose of the photoactivating light that activates, according to photochemical kinetic reactions, a photosensitizer applied to the one or more eyes and generates reactive oxygen species that provide an antimicrobial effect in the one or more eyes, without substantially inducing cross-linking activity that produces biomechanical changes in the one or more eyes.

### BRIEF DESCRIPTION OF THE DRAWINGS

FIG. 1a illustrates a view of an example photoactivation device for photoactivating a photosensitizer in an antimicrobial treatment, according to aspects of the present disclosure.

FIG. 1b illustrates another view of the example photoactivation device of FIG. 1a.

FIG. 2 illustrates a graph of concentration of reactive oxygen species (ROS) generated when various doses of ultraviolet (UV) light are applied to corneas treated with a transepithelial riboflavin formulation, according to aspects of the present disclosure.

FIG. 3 illustrates, corresponding to FIG. 1, a graph of concentration of cross-links generated when the various doses of UV light are applied to corneas treated with the transepithelial riboflavin formulation, according to aspects of the present disclosure.

FIG. 4 illustrates another example photoactivation device for photoactivating a photosensitizer in an antimicrobial treatment, according to aspects of the present disclosure.

FIG. 5 illustrates an example method for employing the example photoactivation device of FIG. 4, according to aspects of the present disclosure.

FIG. 6 illustrates a diagram for photochemical kinetic reactions involving riboflavin and UVA light applied during a corneal treatment, according to aspects of the present disclosure.

### DESCRIPTION

Some antimicrobial treatments (also known as antimicrobial photodynamic therapies) employ photosensitizers to sterilize tissues. In general, when photosensitizers are applied to tissue and exposed to photoactivating illumination, resulting photochemical kinetic reactions can produce antimicrobial agents that place microbes in the tissue under stress and induce an apoptotic or necrotic response in the microbes.

Example antimicrobial treatments may, for instance, employ formulations including various concentrations of riboflavin as a photosensitizer. After a riboflavin formulation is applied to tissue, illumination of the tissue with ultraviolet A (UVA) light in particular results in photochemical kinetic reactions that provide an antimicrobial effect.

In particular, the stroma may be treated with riboflavin, and UVA light is delivered to the cornea to activate the riboflavin in the stroma. Upon absorbing UVA radiation, riboflavin undergoes a reaction with oxygen in which reactive oxygen species (ROS) and other radicals are produced. The ROS can provide an antimicrobial effect in the treated tissue.

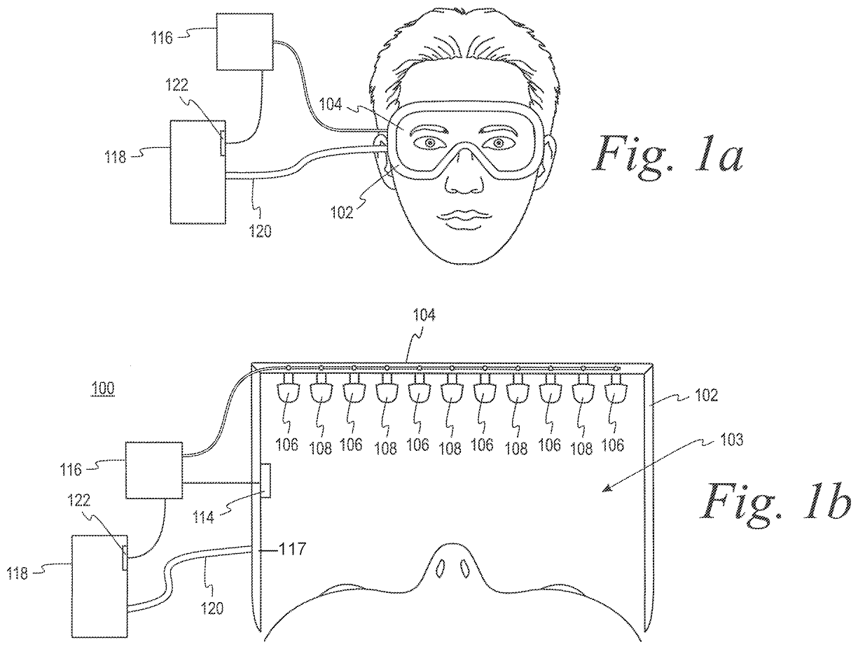

FIGS. 1a, b illustrate an example photoactivation device 100 that is configured to activate a photosensitizer, such as riboflavin, that has been applied to eye tissue according to an antimicrobial treatment. The photoactivation device 100 combines a plurality of features to enhance or otherwise control photochemical kinetic reactions that produce an antimicrobial effect in the targeted eye tissue. The photoactivation device 100 includes a body 102 that defines a substantially closed chamber 103. As shown in FIGS. 1a, b, the photoactivation device 100 fits over the eyes of a subject in a manner similar to eye goggles and may be coupled more securely to the subject's head with a strap, tape, adhesives, and/or the like. The body 102 includes a window 104 formed from glass, plastic, etc., that allows the subject see through the photoactivation device 100. For instance, the window 104 allows the procedure to be monitored and also allows the subject to read, watch television, or be otherwise occupied during the treatment.

The conditions in the chamber 103 can be controlled and monitored to achieve desired photochemical kinetic reactions and to provide an antimicrobial effect in the targeted eye tissue. The photoactivation device 100 includes photoactivating light sources 106 that emit light (e.g., UVA light) to initiate photochemical kinetic reactions with the photosensitizer that has been applied to the targeted eye tissue. The photoactivating light sources 106 may be light emitting diodes (LED's) that can emit selected wavelengths of light, e.g., 365 nm, 450 nm, etc.

The depth and distribution of the antimicrobial effect may be modulated through a timed increase and/or decrease in temperature in the chamber 103 enclosing the targeted eye tissue. Correspondingly, the photoactivation device 100 includes heating elements 108 that generate heat and increase the temperature within the chamber 103. For instance, the heating elements 108 may include LEDs that can emit electromagnetic energy, such as near-infrared (NIR) light, infrared (IR) light, and/or microwaves, to generate heat. Alternatively or additionally, the heating elements 108 may include resistive heating elements or the like. Furthermore, the temperature of the targeted eye tissue may also be decreased by applying chilled gas, evaporative cooling systems, chilled liquids, etc.

A controller 116 is coupled to the photoactivating light sources 106. The controller 116 can control the photoactivating light sources 106 to apply light with any combination/sequence of pulses or continuous wave having any suitable wavelength, power, irradiance, intensity, duration, duty cycle (for pulses), and/or other illumination parameters.

The controller 116 may also be coupled to the heating elements 108 to control the generation of heat. As shown in FIG. 1b, one or more sensors 114 in the photoactivation device 100 may include temperature sensors (e.g., thermostat, optical sensors, etc.) that monitor the temperature in the chamber 103 and provide feedback for the operation of heating elements 108.

The generation of ROS according to the photochemical kinetic reactions may be highly dependent on the oxygen conditions (e.g., concentration, pressure, etc.) in the targeted eye tissue or the environment around the targeted eye tissue. Correspondingly, the photoactivation device 100 can enhance the antimicrobial effect associated with the ROS by controlling the amount of oxygen available during photoactivation of the photosensitizer. The photoactivation device 100 can increase the partial pressure of oxygen in the chamber 103 that encloses the targeted eye tissue. For instance, the partial pressure of the oxygen may be achieved through the use of hyperoxic addition of oxygen up to 100% and/or through hyperbaric pressurization of up to 2 atm.

As shown in FIGS. 1a, b, the photoactivation device 100 includes an inlet 117 that couples the chamber 103 to an oxygen source 118 via a tube 120. Thus, the oxygen source 118 delivers oxygen gas (e.g., humidified oxygen gas) to the chamber 103 to increase the partial pressure of oxygen. The controller 116 may also be coupled to the oxygen source 118 to control the delivery of oxygen gas to achieve the desired concentration of oxygen in the chamber 103. The one or more sensors 114 may also include oxygen sensors to monitor the concentration of oxygen and provide feedback for the operation of the oxygen source 118.

The treated tissue may be exposed to a sequence of different oxygen conditions to generate different amounts of ROS at different depths in the treated tissue. For instance, example antimicrobial treatments may expose the target tissue to normoxic conditions, followed by hyperoxic conditions, and then followed by hyperbaric conditions.

The oxygen gas in the oxygen source 118 has a temperature that may also be controlled by the controller 116. In particular, the oxygen gas may be kept at a lower temperature that allows the oxygen gas to be used as a cooling agent to control the temperature in the chamber 103. The oxygen source 118 includes one or more sensors 122 that measure the temperature of the oxygen gas and provide feedback to manage the temperature of the oxygen gas.

Accordingly, in combination with the photoactivation device 100, the controller 116 can control various aspects of the antimicrobial treatment applied to the targeted eye tissue and achieve more optimal/efficient antimicrobial effects from the photochemical kinetic reactions. In particular, the controller 116 can modulate: (i) the light from the photoactivating light sources 106; (ii) the heat generated by the heating elements 108; (iii) the concentration of oxygen gas in the chamber 103; and/or (iv) the cooling provided by the oxygen gas. The controller 116 can modulate these aspects of the antimicrobial treatment in any combination and sequence of steps. For example, the controller 116 may initially increase the temperature of the treated tissue by generating heat with the heating elements 108 and, after a certain period of time, may cool the treated tissue by applying cooled oxygen gas from the oxygen source 118.

In some embodiments, the window 104 may include a diffuser to allow other external illumination systems to deliver light additionally or alternatively to the treated tissue. Although not shown, aspects of the controller 116 and/or the oxygen source 118 may be coupled to or otherwise combined with the photoactivation device 100 in a single unit.

The one or more sensors 114, 122 provide feedback for modulating these aspects of the antimicrobial treatment. In some cases, additional monitoring can be provided by additional systems. For example, a fluorescence dosimetry system may be employed to determine the distribution/uptake of the photosensitizer as well as the consumption of the photosensitizer during/after the antimicrobial treatment. An example of a fluorescence dosimetry system is described in U.S. Pat. No. 9,020,580, filed Jun. 4, 2012 and titled “Systems and Methods for Monitoring Time Based Photo Active Agent Delivery or Photo Active Marker Presence,” the contents of which are incorporated entirely herein by reference.

The photoactivation device 100 shown in FIGS. 1a, b demonstrates how a device can combine a variety of the features above to enhance or otherwise control photochemical kinetic reactions that produce an antimicrobial effect. Other example embodiments, however, are also contemplated. For instance, photoactivation devices for delivering photoactiving light to corneal tissue are described in U.S. patent application Ser. No. 14/248,966, filed Apr. 9, 2014 and titled “Systems and Methods for Delivering Light in Eye Treatments,” the contents of which are incorporated entirely herein by reference. Such photoactivation devices can be modified to include one or more of the features according to aspects of the present disclosure. For example, the devices can be modified to introduce oxygen gas into the environment of the corneal tissue in a manner similar to the photoactivation device 100.

As the outer-most barrier of the cornea, the epithelium functions to regulate nutrients, including oxygen, that are admitted into the stromal tissue from the tear film. This regulation is carried out via the epithelium's physiological “pumps” that are driven by osmotic pressure across the epithelium due to differential concentrations of barrier-permeable solutes on either side of the epithelium. When healthy, certain nutrients in the tear film that become depleted within the stroma can permeate the epithelium via osmotic pressure to resupply the stroma. However, while oxygen and some other small molecule nutrients can reach the stroma according to this mechanism, certain photosensitizers cannot pass through the epithelium.

Riboflavin, for example, is a relatively large, hydrophilic molecule that cannot penetrate the tight junctions of the epithelium. The epithelium slows the amount of riboflavin that can penetrate the stroma. Thus, a variety of approaches have been employed to overcome low riboflavin diffusivity and deliver sufficient concentrations of riboflavin to the stroma for performing treatments. For some corneal treatments, for instance, the epithelium may be removed (epithelium debridement) before a riboflavin solution is applied directly to the stroma. Although removing the epithelium allows riboflavin to reach the stroma, the approach is associated with patient discomfort, risks of infection, and other possible complications. Furthermore, removing the epithelium may be less appropriate for treatments such as antimicrobial treatments.

Meanwhile, other approaches avoid epithelial debridement. For instance, riboflavin may be provided in a transepithelial formulation that allows riboflavin to pass through the epithelium. In particular, some transepithelial formulations include agents, such as benzalkonium chloride (BAC), with a specific osmolarity of sodium chloride (NaCl). Formulations including BAC are described, for example, in U.S. Patent Application Publication No. 2010/0286156, filed on May 6, 2009, and U.S. Patent Application Publication No. 2013/0267528, filed on Jan. 4, 2013, the contents of these applications being incorporated entirely herein by reference. Other transepithelial formulations may employ other additives, such as ethylenediaminetetraacetic acid (EDTA) or tris(hydroxymethyl)aminomethane (Tris).

Yet other transepithelial formulations may employ non-ionic permeability enhancers. Aspects of using transepithelial formulations with such non-ionic agents are further described further in U.S. Provisional Patent Application No. 62/195,144, filed Jul. 21, 2015, U.S. Provisional Patent Application No. 62/255,452, filed Nov. 14, 2015, U.S. Provisional Patent Application No. 62/262,919, filed Dec. 4, 2015, and U.S. Provisional patent Application No. 62/263,598, filed Dec. 4, 2015, the contents of these applications being incorporated entirely herein by reference.

For instance, such transepithelial formulations employ a non-ionic agent that is chosen using the Hydrophile-Lipophile Balance (HLB) system. The HLB of a permeability enhancer indicates the balance of hydrophilic and lipophilic groups in the molecular structure of the enhancer. Permeability enhancers (or emulsifiers) for the epithelium include a molecule which has both hydrophilic and lipophilic groups. Molecules with HLB number below 9 are considered lipophilic and those above 11 as hydrophilic. Molecules with HLB number between 9 and 11 are intermediate.

For the corneal epithelium, a HLB number that is too great or too small does not help the passage of a photosensitizer through the epithelium. A specific HLB range enhances movement of a photosensitizer through the epithelium. Thus, aspects of the present disclosure employ non-ionic agents that have a hydrophilic/lipophilic balance to achieve optimized diffusivity through the epithelium and the stroma. Advantageously, non-ionic agents are also less corrosive and damaging to the epithelium than ionic agents, such as BAC.

For riboflavin, the HLB range for more effective permeability enhancers has been experimentally determined by the inventors to be between approximately 12.6 and approximately 14.6. A class of permeability enhancers includes various forms of polyethylene glycol (PEG) with different aliphatic chain lengths. According to example embodiments, some riboflavin formulations include specific concentrations of Polidocanol (Polyoxyethylene (9) lauryl ether), which has a HLB number of approximately 13.6.

Some microbes, such as fungi, have dormant phases, while other microbes, such as Acanthamoeba, can create cystic cell membrane barriers. Advantageously, additives that enhance permeability can increase penetration and uptake of photosensitizer by microbes and enhance the antimicrobial effect of the photosensitizer.

Additionally or alternatively, another solution and/or mechanical forces may be applied to enhance the permeability of the epithelium and allow the riboflavin to pass more easily through the epithelium. Examples of approaches for enhancing or otherwise controlling the delivery of a photosensitizer to the underlying regions of the cornea are described, for example, in U.S. Patent Application Publication No. 2011/0288466, filed Apr. 13, 2011, and U.S. Patent Application Publication No. 2012/0289886, filed May 18, 2012, the contents of these applications being incorporated entirely herein by reference.

When photosensitizers (e.g., riboflavin) are applied to the cornea, the subsequent application of photoactivating light (e.g., UVA light) may result in cross-linking activity. In particular, the resulting ROS and/or other radicals further interact with the collagen fibrils to induce covalent bonds that bind together amino acids of the collagen fibrils, thereby cross-linking the fibrils. Such cross-linking activity may be desired for treatments that modify biomechanical properties of the cornea, for instance. For antimicrobial treatments, however, it may be more preferable to generate minimal cross-linking activity while providing the deepest and more predictable generation of ROS for their antimicrobial effect.

Example embodiments may employ the transepithelial formulations described above to deliver a photosensitizer through the epithelium and to desired depths in the corneal tissue. The example embodiments can induce an antimicrobial effect at these depths without inducing cross-linking activity by delivering low doses of photoactivating light that can nevertheless reach these depths and sufficiently generate ROS. In other words, the low doses of photoactivating light minimize cross-linking activity but induce the desired antimicrobial effect. For instance, some implementations may apply UVA light at an irradiance of approximately 0.3 mW/cm² over an extended amount of time to corneal tissue that has been treated with a transepithelial riboflavin formulation with a non-ionic permeability enhancer, such as Polidocanol.

The presence of microbes can be modeled with a molar concentrator, and the killing efficiency can be represented by the concentration of microbes multiplied by a susceptibility constant for each type of microbe. Additionally, for riboflavin, a model based on the photochemical kinetic reactions described herein may be modified to include an additional molar concentration of microbes. In this manner, the killing efficiency can be calculated and validated by experiment. The total number of photoreactive sites in molar concentration is the sum of two concentrations, microbe molar concentration plus cross-linking site concentration.

By applying a low dose of photoactivating light over an extended amount of time, ROS are generated at desired depths and at rates that allow the whole thickness of the cornea to reach the killing threshold at once while minimizing cross-linking of the anterior cornea.

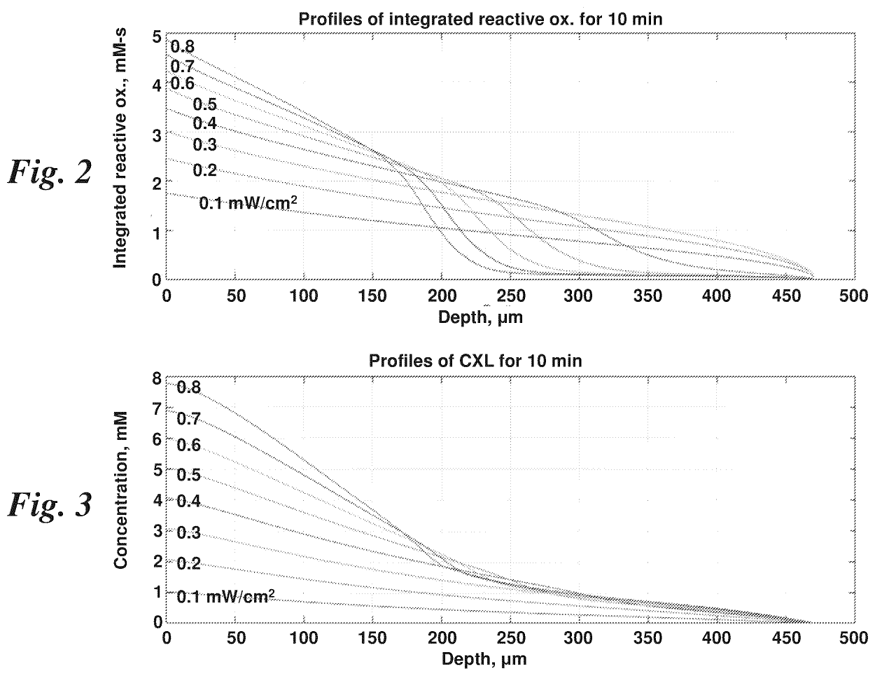

FIGS. 2 and 3 illustrate respective graphs of concentrations for ROS and cross-links generated when various doses of UVA light are applied to corneas treated with a transepithelial riboflavin formulation with a non-ionic permeability enhancer as described above. In particular, the doses of UVA light are applied at irradiances of 0.1 mW/cm². 0.2 mW/cm². 0.3 mW/cm². 0.4 mW/cm². 0.5 mW/cm². 0.6 mW/cm². 0.7 mW/cm², and 0.8 mW/cm² for 10 minutes. As shown in FIGS. 2 and 3, for instance, the results from an irradiance of 0.3 mW/cm² are predominately dictated by Beer's law and full oxygen depletion is never achieved for the full stromal thickness as seen with the greater doses. The ROS concentration profile as a function of depth is maintained with the irradiance of 0.3 mW/cm² but increases with the greater irradiances. Therefore, an antimicrobial threshold can be achieved to a given depth for a given concentration of microbes.

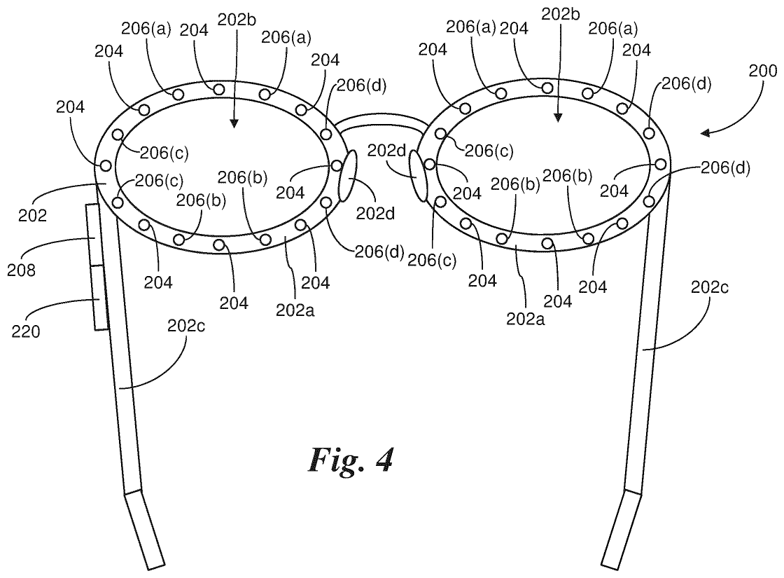

FIG. 4 illustrates another photoactivation device 200 that is configured to activate a photosensitizer, such as riboflavin, that has been applied to eye tissue according to an antimicrobial treatment. As described above, when photoactivated, the photosensitizer generates ROS that provides an antimicrobial effect. The eye tissue may be treated with a transepithelial formulation with a non-ionic permeability enhancer as described above.

According to one implementation, a medical practitioner, e.g., a nurse, or the patient (once instructed) places drops of the transepithelial photosensitizer formulation every 30 to 60 seconds for a period of approximately 15 to 20 minutes. The transepithelial photosensitizer formulation can be applied to the eyes without the use of specula.

As shown in FIG. 4, the photoactivation device 200 includes a body 202. The body 202 may be shaped and worn like an eyeglasses frame. As such, the body 202 includes rims 202a defining openings 202b allowing the subject can see through the body 202. The body 202 also includes temples 202c and nosepads 202d that can support the body 202 on the head of the subject. Although the photoactivation device 200 in FIG. 4 resembles a pair of eyeglasses, it is understood that other shapes and configurations may be employed to situate the photoactivation device 200 stably about the eyes.

The body 202 includes a plurality of photoactivating light sources 204 that can direct photoactivating light to each eye of the subject from the top, bottom, left, and right. In some cases, the photoactivating light sources 204 may include light-emitting diodes (LED's) that direct UVA light simultaneously to eyes that have been treated with a riboflavin formulation. The number of photoactivating light sources 204 may be limited to the number required to provide the desired low dose of photoactivating light, e.g., delivered at an irradiance of approximately 0.3 mW/cm² for approximately at least 10 minutes.

The body 202 also includes a plurality of guide light sources 206 that emit visible light from above, below, left, and right of each eye. In some cases, the guide light sources 206 may include LEDs. At least one top guide light source 206 (a) emits light from above each eye; at least one bottom guide light source 206 (b) emits light from below each eye; at least one left guide light source 206 (c) emits light from the left of each eye; and at least one right guide light source 206 (d) emits light from the right of each eye.

A controller 208, e.g., in the form of an electronic/electric chip/circuit, is coupled to the guide light sources 206. The controller 208 can alternately illuminate the guide light sources 206. In particular, the controller 208 may repeatedly, in series: (1) illuminate the top guide light source(s) 206 (a) for a predetermined period of time (e.g., 10 seconds or other optimal period) while the other guide light sources 206 (b), (c), (d) remain off; (2) illuminate the bottom guide light source(s) 206 (b) for the predetermined period of time while the other guide light sources 206 (a), (c), (d) remain off; (3) illuminate the left guide light source(s) 206 (c) for the predetermined period of time while the other guide light sources 206 (a), (b), (d) remain off; and illuminate the right guide light source(s) 206 (d) for the predetermined period of time while the other guide light sources 206 (a), (b), (c) remain off.

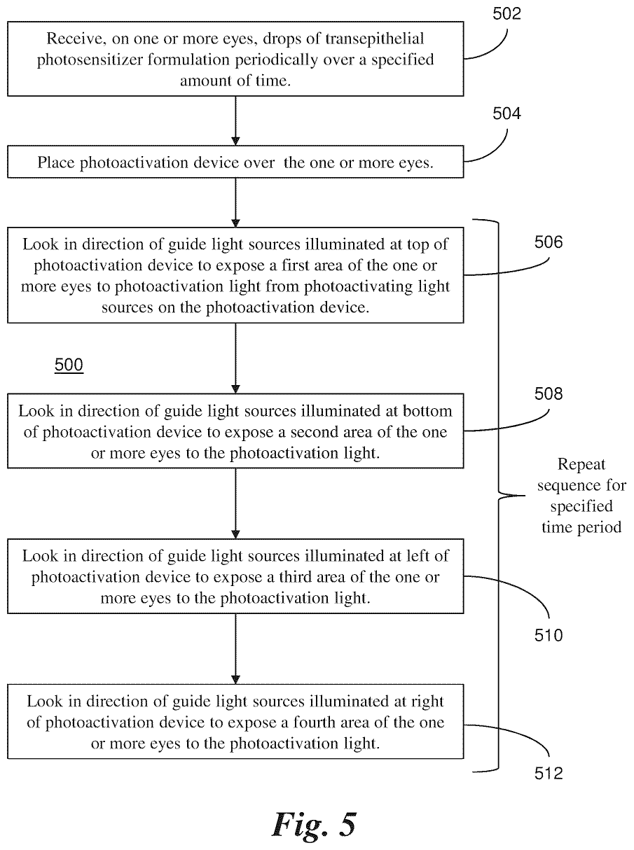

FIG. 5 illustrates a method 500 that corresponds to the example above. In step 502, the one or more eyes receive drops of transepithelial photosensitizer formulation periodically over a specified amount of time. In step 504, the photoactivation device 200 is placed over the one or more eyes. In step 506, the one or more eyes look at the guide light sources 206 (a) illuminated at the top of the photoactivation device 200 to expose a first area of the one or more eyes to photoactivation light from photoactivating light sources 204 on the photoactivation device 200. In step 508, the one or more eyes look at the guide light sources 206 (b) illuminated at the bottom of the photoactivation device 200 to expose the one or more eyes to the photoactivation light. In step 510, the one or more eyes look at the guide light sources 206 (c) illuminated at the left of the photoactivation device 200 to expose a third area of the one or more eyes to the photoactivation light. In step 512, the one or more eyes look at the guide light sources 206 (d) illuminated at the right of the photoactivation device 200 to expose a fourth area of the one or more eyes to the photoactivation light. In alternative embodiments, the guide light sources 206 may be alternately illuminated in a different sequence. Moreover, the guide light sources 206 may direct light to the eyes from additional directions, e.g., top-left, top-right, bottom-left, bottom-right, etc.

Accordingly, the patient Is directed to move his or her eyes to follow the alternately illuminated guide light sources 206 (i.e., up, down, to the left, to the right, and so on), thereby moving different areas of the eye, e.g., corneal surface, to the open area between the top and bottom eyelids. Even with blinking, substantially the entire surface of each eye is exposed between the top and bottom eyelids to the photoactivating light from the photoactivating light sources 204, and the photosensitizer in the treated tissue can be photoactivated for the antimicrobial effect. In this way, substantially the entire eye surface gets full coverage of irradiance without the need for specula to force the eyes wide open for the delivery of photoactivating light. The patient may sit up or lay down for the procedure for as long as necessary. Because the irradiance is low and the procedure lasts for an extended amount of time, irradiance variation is averaged and greatly minimized over time.

The body 202 also includes a battery 210 to power the photoactivating light sources 204, the guide light sources 206, and the controller 208. Initially, a plastic pull-tab can electrically separate the battery 210 from the other components. When the photoactivation device 200 is needed to deliver photoactivating light to the treated eyes, the pull-tab can be removed to connect the battery 210 with a conductive contact which delivers electrical power to the other components. Alternatively, the frame 202 may include an electrical switch that can be selectively operated to connect the battery 210 with the other components. The power from the battery 210 may be limited to what is necessary to operate the photoactivating light sources 204 and the guide light sources 206 to deliver the photoactivating light to the entire ocular surface with the desired low irradiance and desired extended irradiation time.

The end of the treatment may coincide with the depletion of power from the battery 210. Alternatively, the controller 208 may control the irradiation time. Alternatively, the components of the photoactivation device 200 may turn off (e.g., burn out) and self-destruct after a given amount of irradiation time.

Due to the configuration above, the photoactivation device 200 may be employed as a single-use, disposable device. The photoactivation device 200 does not include any lenses and can be inexpensively manufactured. For instance, the body 202 may be molded from plastic. Because photoactivation device 200 is not positioned too close to the eyes (e.g., the surgical field), the photoactivation device 200 should be clean but does not necessarily have to be sterile. Furthermore, the photoactivation device 200 might not be considered medical waste and as such may not require special disposal procedures.

The photoactivation device 200 may be configured to become inoperable once the treatment is complete. For instance, the battery 210 cannot be replaced once the power is depleted and the treatment is complete. Additionally or alternatively, as described above, the components of the photoactivation device 200 may self-destruct after a given amount of irradiation time.

In general, the photoactivation device 200 is more convenient and cost-effective than other irradiation systems. As such, the photoactivation device 200 may be more feasible for treatments in the third world and/or other remote locations.

The use of the photoactivation device 200 is not limited to humans. Indeed, the photoactivation device 200 can be especially modified/configured for treatment of animals, such as dogs, cats, horses, etc.

As described above, photochemical kinetic reactions can produce antimicrobial agents that place microbes in the tissue under stress and induce an apoptotic or necrotic response in the microbes. A description of photochemical kinetic reactions for riboflavin is provided, for example, in International Patent Application No. PCT/US15/57628, filed Oct. 27, 2015, the contents of which are incorporated entirely herein by reference. When riboflavin absorbs radiant energy, especially UVA light, it undergoes photoactivation. There are two photochemical kinetic pathways for riboflavin photoactivation, Type I and Type II. Some of the reactions involved in both the Type I and

Type II mechanisms are as follows:

Common reactions:

| | | |
|---|---|---|
| Rf → Rf₁*, | I; | (r1) |
| Rf₁* → Rf, | k1; | (r2) |
| Rf₁* → Rf₃*, | k2; | (r3) |

Type I reactions:

| | | |
|---|---|---|
| Rf₃* + DH → RfH⁻ + D⁻, | k3; | (r4) |
| 2RfH → Rf + RfH₂, | k4; | (r5) |

Type II reactions:

| | | | |
|---|---|---|---|
| Rf₃* + O₂ → Rf + O₂¹, | k5; | | (r6) |
| DH + O₂¹ → Dₒₓ, | k6; | | (r7) |
| Dₒₓ + DH → D − D, | k7; | CXL | (r8) |

In the reactions described herein, Rf represents riboflavin in the ground state.

Rf*₁ represents riboflavin in the excited singlet state. Rf*₃ represents riboflavin in a triplet excited state. Rf*^{- is the reduced radical anion form of riboflavin. RfH* is the radical form of riboflavin. RfH}₂ is the reduced form of riboflavin. DH is the substrate. DH*⁺ is the intermediate radical cation. D* is the radical. Dₒₓ is the oxidized form of the substrate.

Riboflavin is excited into its triplet excited state Rf*₃ as shown in reactions (r1) to (r3). From the triplet excited state Rf*₃, the riboflavin reacts further, generally according to Type I or Type II mechanisms. In the Type I mechanism, the substrate reacts with the excited state riboflavin to generate radicals or radical ions, respectively, by hydrogen atoms or electron transfer. In Type II mechanism, the excited state riboflavin reacts with oxygen to form singlet molecular oxygen. The singlet molecular oxygen then acts on tissue to produce additional cross-linked bonds.

Oxygen concentration m the cornea is modulated by UVA irradiance and temperature and quickly decreases at the beginning of UVA exposure. Utilizing pulsed light of a specific duty cycle, frequency, and irradiance, input from both Type I and Type II photochemical kinetic mechanisms can be employed to achieve a greater amount of photochemical efficiency. Moreover, utilizing pulsed light allows regulating the rate of reactions involving riboflavin. The rate of reactions may either be increased or decreased, as needed, by regulating, one of the parameters such as the irradiance, the dose, the on/off duty cycle, riboflavin concentration, soak time, and others. Moreover, additional ingredients that affect the reaction and cross-linking rates may be added to the cornea.

If UVA radiation is stopped shortly after oxygen depletion, oxygen concentrations start to increase (replenish). Excess oxygen may be detrimental in the corneal cross-linking process because oxygen is able to inhibit free radical photopolymerization reactions by interacting with radical species to form chain-terminating peroxide molecules. The pulse rate, irradiance, dose, and other parameters can be adjusted to achieve a more optimal oxygen regeneration rate. Calculating and adjusting the oxygen regeneration rate is another example of adjusting the reaction parameters to achieve a desired amount of corneal stiffening.

Oxygen content may be depleted throughout the cornea, by various chemical reactions, except for the very thin corneal layer where oxygen diffusion is able to keep up with the kinetics of the reactions. This diffusion-controlled zone will gradually move deeper into the cornea as the reaction ability of the substrate to uptake oxygen decreases.

Riboflavin is reduced (deactivated) reversibly or irreversibly and/or photo-degraded to a greater extent as irradiance increases. Photon optimization can be achieved by allowing reduced riboflavin to return to ground state riboflavin in Type I reactions. The rate of return of reduced riboflavin to ground state in Type I reactions is determined by a number of factors. These factors include, but are not limited to, on/off duty cycle of pulsed light treatment, pulse rate frequency, irradiance, and dose. Moreover, the riboflavin concentration, soak time, and addition of other agents, including oxidizers, affect the rate of oxygen uptake. These and other parameters, including duty cycle, pulse rate frequency, irradiance, and dose can be selected to achieve more optimal photon efficiency and make efficient use of both Type I as well as Type II photochemical kinetic mechanisms for riboflavin photosensitization. Moreover, these parameters can be selected in such a way as to achieve a more optimal chemical amplification effect.

In addition to the photochemical kinetic reactions (r1)-(r8) above, however, the present inventors have identified the following photochemical kinetic reactions (r9)-(r26) that also occur during riboflavin photoactivation:

| | | | |
|---|---|---|---|
| Rf₃* → Rf, | k8; | | (r9) |
| Rf₃* + Rf → 2RfH′, | k9; | | (r10) |
| RfH₂ + O₂ → RfH′ + H⁺ + O₂⁻, | k10 | | (r11) |
| RfH + O₂ → Rf + H⁺ + O₂⁻, | k11 | | (r12) |
| 2 RfH₂ + O₂⁻ → 2 RfH + H₂O₂, | k12 | | (r13) |
| 2 RfH + O₂⁻ → 2 Rf + H₂O₂, | k13 | | (r14) |
| RfH + H₂O₂ → OH + Rf + H₂O, | k14 | | (r15) |
| OH + DH → D + H₂O, | k15 | | (r16) |
| D + D → D − D, | k16 | CXL | (r17) |
| O₂¹ → O₂, | k18 | | (r18) |
| D + RfH₂ → RfH + DH, | k19 | | (r19) |
| $Rf + Rf \begin{matrix}{x}_{a}^{+} \\ \to \\ ← \\ {x}_{a}^{-}\end{matrix} {A}_{1} ,$ | kₐ = kₐ⁺/ₐ⁻ | | (r20) |
| ${Rf H}_{2} + {Rf H}_{2} \begin{matrix}{x}_{a}^{+} \\ \to \\ ← \\ {x}_{a}^{-}\end{matrix} {A}_{2} ,$ | kₐ = kₐ⁺/ₐ⁻ | | (r21) |
| $Rf + {Rf H}_{2} \begin{matrix}{x}_{b}^{+} \\ \to \\ ← \\ {x}_{b}^{-}\end{matrix} {A}_{2} ,$ | | | (r22) |
| k_{b} = k_{b}⁺/_{b}⁻ | | | |
| Rf₁* + A → Rf + A, | k₁ₐ | | (r23) |
| Rf₃* + A → Rf + A, | k₃ₐ | | (r24) |
| 2 O₂⁻ → O₂ + H₂O₂, | k₁₂ | | (r25) |
| OH + CXL → insert products, | K_{OH} | | (r26) |

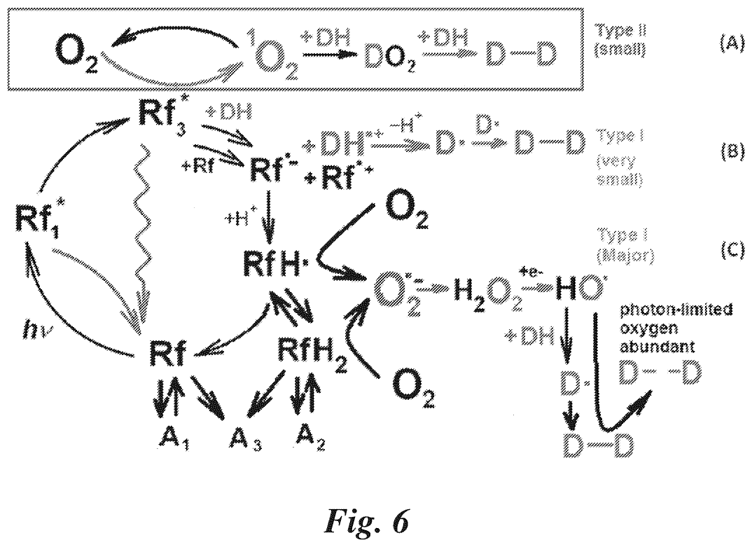

FIG. 6 illustrates a diagram for the photochemical kinetic reactions provided in reactions (r1) through (r26) above. The diagram summarizes photochemical transformations of riboflavin (Rf) under UVA photoactivating light and its interactions with various donors (DH) via electron transfer. As shown, cross-linking activity occurs: (A) through the presence of singlet oxygen in reactions (r6) through (r8) (Type II mechanism); (B) without using oxygen in reactions (r4) and (r17) (Type I mechanism); and (C) through the presence of peroxide (H₂O₂), superoxide (O₂″), and hydroxyl radicals (OH) in reactions (r13) through (r17).

As shown in FIG. 6, the present inventors have also determined that the cross-linking activity is generated to a greater degree from reactions involving peroxide, superoxide, and hydroxyl radicals. Cross-linking activity is generated to a lesser degree from reactions involving singlet oxygen and from non-oxygen reactions. Some models based on the reactions (r1)-(r26) may account for the level of cross-linking activity generated by the respective reactions. For instance, where singlet oxygen plays a smaller role in generating cross-linking activity, models may be simplified by treating the cross-linking activity resulting from singlet oxygen as a constant.

All the reactions start from Rf₃* as provided in reactions (r1)-(r3). The quenching of Rf₃* occurs through chemical reaction with ground state Rf in reaction (r10), and through deactivation by the interaction with water in reaction (r9).

Excess oxygen may be detrimental in corneal cross-linking process. As shown in FIG. 6, when the system becomes photon-limited and oxygen-abundant, cross-links can be broken from further reactions involving superoxide, peroxide, and hydroxyl radicals. Indeed, in some cases, excess oxygen may result in net destruction of cross-links versus generation of cross-links.

A large variety of factors as described herein affect the rate of the cross-linking reaction and the amount of biomechanical stiffness achieved due to cross-linking. A number of these factors are interrelated, such that changing one factor may have an unexpected effect on another factor. However, a more comprehensive model for understanding the relationship between different factors for riboflavin treatment is provided by the photochemical kinetic reactions (r1)-(r26) identified above. Accordingly, systems and methods can adjust various parameters for photosensitizer treatment according to this photochemical kinetic model, which provides a unified description of oxygen dynamics and cross-linking activity. The model can be employed to evaluate expected outcomes based on different combinations of treatment parameters and to identify the combination of treatment parameters that provides the desired result. The parameters, for example, may include, but is not limited to: the concentration(s) and/or soak times of the applied photosensitizer; the dose(s), wavelength(s), irradiance(s), duration(s), and/or on/off duty cycle(s) of the photoactivating light; the oxygenation conditions in the tissue; and/or presence of additional agents and solutions.

As further described above, example embodiments can generate ROS at desired depths and at rates to achieve an antimicrobial effect throughout the thickness of the cornea while minimizing cross-linking of the anterior cornea. The photochemical kinetic reactions above can be employed to determine the threshold at which cross-linking activity is generated at depths within the cornea. Using a model based on the photochemical kinetic reactions, the example embodiments can be configured accordingly to generate ROS for the antimicrobial effect without reaching the determined threshold for cross-linking activity.

It is understood, however, that alternative embodiments may call for cross-linking activity (to modify biomechanical properties) in addition to antimicrobial treatment. As such, the model based on the photochemical kinetic reactions allows these alternative embodiments to generate ROS and/or other radicals for the desired antimicrobial effect and desired cross-linking activity.

In addition to the factors described above, example embodiments may enhance the photochemical kinetic reactions by adding a metal, such as iron or copper, to the riboflavin formulation. A description of how additives can affect photochemical kinetic reactions is provided, for example, in U.S. patent application Ser. No. 14/281,638, filed May 19, 2014 and titled “Systems, Methods, and Compositions for Cross-Linking” and U.S. Provisional Patent Application No. 62/086,572, filed Dec. 2, 2014 and also titled “Systems, Methods, and Compositions for Cross-Linking,” the contents of these application being incorporated entirely herein by reference.

For instance, trace amounts of copper (ranging from approximately 0.1 mM to approximately 10 mM) can provide an enhanced antimicrobial effect for a riboflavin formulation. Copper can enhance the photodynamic effect of riboflavin through a Fenton-type reaction. Moreover, copper on its own (specifically, copper ions) can have an antimicrobial effect even when it is not combined with a photosensitizer. Therefore, the enhanced mode of action for a riboflavin formulation with a copper additive involves enhancement though the Fenton-type reaction and/or the antimicrobial effect of the copper by itself.

The photochemical kinetic reactions for a riboflavin formulation can be enhanced by adding a deuterated water (D2O), also known as “heavy water.” D₂O by itself does not kill bacteria. Skladnev D. A., et al. Methylotrophic Bacteria as Sources of 2H- and 13C-amino Acids. Biotechnology (1996), pp. 14-22. However, D₂O can increase the presence of singlet oxygen when used in combination with a photosensitizer formulation (and optionally other additives). Singlet oxygen is one of the ROS responsible for producing an antimicrobial effect through Type II photochemical kinetic energy transfer. Type II photochemical kinetic reactions are described, for example, in U.S. patent application Ser. No. 13/841,617 cited above. Thus, example embodiments may employ D2O to enhance the antimicrobial effect associated with singlet oxygen.

Example embodiments may also employ timed application of agents, such as dimethyl sulfoxide (DMSO), which can cause penetration of a photosensitizer to desired depths in the targeted tissue and produce an antimicrobial effect at the desired depths. In some cases, the antimicrobial effect at the desired depths may be further enhanced by increasing the oxygen concentration available for the photochemical reactions with the photosensitizer.

Example embodiments may also employ timed application of quenching agents to generate greater antimicrobial effect at the desired depths. The quenching agents can limit the photochemical reaction in regions closer to the surface of the tissue and allow the antimicrobial effect of the photosensitizer to take place deeper in the tissue. Quenching agents are described, for example, in U.S. patent application Ser. No. 13/475,175, filed May 18, 2012 and titled “Controlled Application of Cross-Linking Agent,” the contents of which are incorporated entirely herein by reference.

Example embodiments may also increase or decrease the pH of the tissue to enhance the antimicrobial effect of antimicrobial treatments. The pH of the tissue may be modified by selectively applying acidic or basic solutions. In some cases, the acidic or basic solutions may include additives as described herein. For example, the solutions may include quenching agents to control the photochemical reactions at a given depth within the tissue.

Example embodiments may employ a dispensing device configured to apply different formulations and/or different concentrations according to a predetermined sequence. The dispensing device, for instance, may be a charged nanocloud device that applies the photosensitizer formulations via aerosolized electro-spraying. In some cases, the dispensing device may generate and deliver a dual payload of ionized ROS encapsulated in photosensitizer nanoparticles for simultaneous intra-stromal deposition.

Example embodiments may additionally employ water nanoparticles for antimicrobial applications. Electro-spraying ionized water (“engineered water”) results in nano-caging ROS via an excess of electrons loaded during droplet fission thereby conferring the nanoparticles with antimicrobial properties.

Example embodiments may employ nanostructures to promote delivery of photosensitizer formulations to the target tissue and enhance the antimicrobial effect of the photochemical kinetic reactions. The nanostructures may include, but are not limited to, liposomes, polymeric micelles, ceramic (graphene oxide) and metallic nanorods. These nanostructures may be included in drops that are applied to the target tissue. Alternatively, specially configured structures may be employed to allow the nanostructures and photosensitizer formulations to penetrate the target tissue.

For instance, a contact lens device may be configured to allow different photosensitizer formulations and nanostructures to penetrate a cornea in a corneal procedure. The contact lens device may be applied to the cornea for several minutes or even a few hours before illumination is applied to initiate photochemical kinetic reactions. Such a device facilitates delivery through the epithelium for procedures that keep the epithelium in place, i.e., “epi-on” procedures.

In an example procedure, very low concentrations of drug formulation may be applied to eye tissue, followed by application of illumination at very low irradiance levels, e.g., a formulation with approximately 0.02% riboflavin concentration followed by approximately 1 mW/cm² illumination of UV light. The contact lens devices described above can be applied to the subject's eyes for 30-90 minutes to deliver the formulation. Once the contact lens devices are removed and the photoactivation device 100 is positioned over the subject's eyes to deliver the illumination to generate the photochemical kinetic reactions.

In view of the foregoing, example embodiments can enhance antimicrobial treatments by any combination of:
Employing different photosensitizer formulations at various concentrations;
employing specialized additives with the photosensitizer formulation(s);
controlling oxygen available to the photochemical reactions through hyperbaric, hyperoxic, and/or hypoxic conditions;
employing time dependent quenching agents;
manipulating the pH of the photosensitizers or additives;
employing nanostructures;
controlling the delivery of photosensitizer(s) to the tissue; and/or controlling the delivery of light to the target tissue treated with the photosensitizer formulation(s).

Although the example embodiments above involve treatments of the eye, it is understood that aspects of the present disclosure can be applied to treatments of other parts of the body. For instance, alternative applications such as Ventilator Associated Pneumonia (VAP) treatments can be addressed by the use of combinations of acrosolized drugs/photosensitizers and ionized ROS in water nanoparticles. These can be delivered to the oral and tracheal regions with targeted multi-drug resistant anti-bacterial payloads (MDR A. baumannii, P. aeruginosa). In an example VAP application, a pre-tracheal mouthpiece tube having actinic illumination targets ROS-photosensitizer nanoparticles flowing into the oral cavity.

The embodiments described herein may employ controllers and other devices for processing information and controlling aspects of the example systems. For example, the example photoactivation device 100 shown in FIGS. 1a, b includes the controller 116 or the photoactivation device 200 shown in FIG. 4 includes the controller 208. Generally, the controllers include one or more processors. The processor(s) of a controller or other devices may be implemented as a combination of hardware and software elements. The hardware elements may include combinations of operatively coupled hardware components, including microprocessors, memory, signal filters, electronic/electric chip/circuit, etc. The processors may be configured to perform operations specified by the software elements, e.g., computer-executable code stored on computer readable medium. The processors may be implemented in any device, system, or subsystem to provide functionality and operation according to the present disclosure. The processors may be implemented in any number of physical devices/machines. Indeed, parts of the processing of the example embodiments can be distributed over any combination of processors for better performance, reliability, cost, etc.

The physical devices/machines can be implemented by the preparation of integrated circuits or by interconnecting an appropriate network of conventional component circuits, as is appreciated by those skilled in the electrical art(s). The physical devices/machines, for example, may include field programmable gate arrays (FPGA's), application-specific integrated circuits (ASIC's), digital signal processors (DSP's), etc.

Appropriate software can be readily prepared by programmers of ordinary skill based on the teachings of the example embodiments, as is appreciated by those skilled in the software arts. Thus, the example embodiments are not limited to any specific combination of hardware circuitry and/or software. Stored on one computer readable medium or a combination of computer readable media, the computing systems may include software for controlling the devices and subsystems of the example embodiments, for driving the devices and subsystems of the example embodiments, for enabling the devices and subsystems of the example embodiments to interact with a human user (user interfaces, displays, controls), etc. Such software can include, but is not limited to, device drivers, operating systems, development tools, applications software, etc. A computer readable medium further can include the computer program product(s) for performing all or a portion of the processing performed by the example embodiments. Computer program products employed by the example embodiments can include any suitable interpretable or executable code mechanism, including but not limited to complete executable programs, interpretable programs, scripts, dynamic link libraries (DLLs), applets, etc. The processors may include, or be otherwise combined with, computer-readable media. Some forms of computer-readable media may include, for example, a hard disk, any other suitable magnetic medium, any suitable optical medium, RAM, PROM, EPROM, flash memory, any other suitable memory chip or cartridge, any other suitable non-volatile memory, a carrier wave, or any other suitable medium from which a computer can read.

The controllers and other devices may also include databases for storing data. Such databases may be stored on the computer readable media described above and may organize the data according to any appropriate approach. For example, the data may be stored in relational databases, navigational databases, flat files, lookup tables, etc.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

1. A wearable treatment system, comprising:
a body configured to be positioned on a head of a subject;
one or more photoactivating ultraviolet light sources coupled to the body; and
a controller including one or more processors and one or more computer readable media, characterized in that:
the wearable treatment system further comprises a plurality of guide light sources coupled at a plurality of positions about the body and configured to direct visible light to the two eyes from a plurality of directions,
the body is shaped to be positioned over two eyes of the subject, the body including one or more windows or openings that allow the two eyes to see through the body,
the one or more processors of the controller are configured to execute instructions from the computer readable media to operate the one or more photoactivating ultraviolet light sources, and to execute instructions from the computer readable media to operate the guide light sources to alternately direct the visible light from each direction according to a sequence wherein the subject is directed to look, with the two eyes, in each direction according to the sequence and different respective areas of the two eyes are exposed to the ultraviolet light from the one or more photoactivating ultraviolet light sources.

2. The system of claim 1, wherein the one or more ultraviolet light sources deliver the ultraviolet light as at least one of pulses or continuous wave.

3. The system of claim 1, wherein the one or more photoactivating light sources to deliver photoactivating light to the eye according to illumination parameters that determine a dose of the photoactivating light.

4. The system of claim 3, wherein the illumination parameters include at least one of wavelength, power, irradiance, intensity, duration, or duty cycle.

5. The system of claim 3, wherein the one or more processors of the controller are further configured to execute instructions from the computer readable media to determine the illumination parameters based on a model of photochemical kinetic reactions.

6. The system of claim 1, further including one or more heating elements to generate heat at a selected region of one or more of the eyes.

7. The system of claim 6, wherein the one or more heating elements are coupled to the body.

8. The system of claim 6, wherein the one or more processors are further configured to execute instructions from the computer readable media to operate the one or more heating elements.

9. The system of claim 1, wherein the body defines a chamber shaped to be positioned over and enclose the two eyes.

10. The system of claim 1, further including an oxygen source configured to provide oxygen to a selected region of one or more of the eyes.

11. The system of claim 10, wherein the one or more processors are further configured to execute instructions from the computer readable media to operate the oxygen source.

12. The system of claim 10, wherein the body defines a chamber shaped to be positioned over and enclose the two eyes and includes an inlet configured to couple the body to the oxygen source, the chamber receiving the oxygen from the oxygen source via the inlet to modify oxygen conditions in the chamber.

13. The system of claim 12, wherein the oxygen from the oxygen source modifies a temperature in the chamber according to temperature parameters.

14. The system of claim 10, wherein the oxygen from the oxygen source modifies a temperature at the selected region of one or more of the eyes.

15. The system of claim 1, wherein the body is shaped as a frame for eyeglasses including rims, temples, and nosepads, the rims defining the openings, and the temples and nosepads supporting the body on the head of the subject.

16. The system of claim 1, further comprising a battery coupled to the one or more ultraviolet light sources, wherein the ultraviolet light is directed according to a duration determined by a battery life of the battery.

17. The system of claim 1, further comprising applying one or more permeability enhancers to the eye.

18. The system of claim 17, wherein the one or more permeability enhancers include non-ionic surfactants.

19. The system of claim 1, wherein the photoactivating light activates a photosensitizer.

20. The system of claim 19, wherein the photosensitizer includes riboflavin.
